(19) United States
(12) Patent Application Publication
(10) Pub. No.: US 2024/0285842 A1
(43) Pub. Date: Aug. 29, 2024

(54) DRAINAGE SYSTEM INCLUDING FLUID COLLECTION APPARATUS HAVING A SELECTABLE NEGATIVE PRESSURE FOR DRAINING AND COLLECTING BODILY FLUIDS FROM A BODY CAVITY

(71) Applicant: BARD PERIPHERAL VASCULAR, INC., Franklin Lakes, NJ (US)

(72) Inventor: RAJASEKARA PRABHU, Thiruchuli (IN)

(73) Assignee: BARD PERIPHERAL VASCULAR, INC., Franklin Lakes, NJ (US)

(21) Appl. No.: 18/572,304

(22) PCT Filed: Jun. 22, 2021
(86) PCT No.: PCT/US21/38388

§ 371 Date: Dec. 20, 2023

(51) Int. Cl. A61M 1/00

(52) U.S. Cl. CPC A61M 1/684; A61M 1/60; A61M 2210/101

(57) ABSTRACT

A drainage system for draining bodily fluid from a body cavity includes a fluid-receiving body having a fluid-receiving chamber located therein that receives a bodily fluid through an inlet. An actuation body is connected to the fluid-receiving body. The actuation body includes an actuation member that is accessible from outside the actuation body and is manually movable relative to the actuation body along a dimension of the actuation body. The actuation member is operatively connected to a suction inducing member. Manual actuation of the actuation member relative to the actuation body moves the suction inducing member thereby increasing a volume of a suction chamber and reducing a pressure within the suction chamber. The reduced pressure communicated through the inlet of the fluid-receiving body.

### TECHNICAL FIELD

The present specification generally relates to drainage systems and methods for draining bodily fluid from body cavities and, more specifically, to drainage systems and methods including fluid collection apparatuses for draining and collecting bodily fluids.

### BACKGROUND

The pleural cavity is the thin, fluid-filled space between the two pulmonary pleurae (known as visceral and parietal) of each lung. A pleura is a serous membrane that folds back onto itself to form a two-layered membranous pleural sac. The plural space is normally filled with approximately five to 20 mL of serous fluid. The turnover of fluid in the pleural space is normally quite rapid-roughly 35 to 75% of the total fluid per hour, so that 5 to 10 L of fluid move through the pleural space each day. A pleural effusion is a build-up of fluid in the pleural space. Pleural effusion may also be referred to as effusion or pulmonary effusion. The type of fluid that forms a pleural effusion may be categorized as transudate or exudate.

A disruption in the balance between the movement of fluid into the pleural space and the movement of fluid out of the pleural space may produce fluid accumulation in the pleural space. Such disruptions may include, for example, (1) increased capillary permeability resulting from inflammatory processes such as pneumonia, (2) increased hydrostatic pressure as in congestive heart failure, (3) increased negative intrapleural pressure as seen in atelectasis (partial or total lung collapse), (4) decreased oncotic pressure as occurs in the nephrotic syndrome with hypoalbuminemia, and (5) increased oncotic pressure of pleural fluid as occurs in the inflammation of pleural tumor growth or infection. Pleural effusion is particularly common in patients with disseminated breast cancer, lung cancer or lymphatic cancer and patients with congestive heart failure, but also occurs in patients with nearly all other forms of malignancy.

There are a number of drainage systems for managing recurrent pleural effusions and malignant ascites at home. One such system is the PleurX™ drainage system, commercially available from Becton, Dickinson and Company. The PleurX™ drainage system includes a drainage catheter and drainage bottles that collect fluid. The drainage catheter is inserted, typically as a simple outpatient procedure, in the chest for draining pleural effusions or in the abdomen for malignant ascites. An end of the drainage catheter stays outside of the body, hidden under a bandage when not in use. To drain, the end of the drainage catheter is connected to a drainage line on the bottle that is pre-evacuated to provide a negative pressure, and the negative pressure inside the bottle is used to automatically draw out the bodily fluid.

Accordingly, here is a need for a drainage system that includes a fluid collection device having a selectable negative pressure for draining and collecting bodily fluid.

### SUMMARY

According to a first embodiment, a drainage system for draining bodily fluid from a body cavity includes a fluid-receiving body having a fluid-receiving chamber located therein that receives a bodily fluid through an inlet. An actuation body is connected to the fluid-receiving body. The actuation body includes an actuation member that is accessible from outside the actuation body and is manually movable relative to the actuation body along a dimension of the actuation body. The actuation member is operatively connected to a suction inducing member. Manual actuation of the actuation member relative to the actuation body moves the suction inducing member thereby increasing a volume of a suction chamber and reducing a pressure within the suction chamber. The reduced pressure communicated through the inlet of the fluid-receiving body.

According to another embodiment, a method of forming a drainage system comprising a suction chamber configured to provide reduced pressure that is communicated to a body cavity for removing bodily fluids from the body cavity is provided. The method includes forming an actuation body and a fluid-receiving body. The fluid-receiving body has a fluid-receiving chamber located therein that receives a bodily fluid through an inlet. An actuation member and a suction inducing member connected thereto are movably connected to the actuation body. The fluid-receiving body has a fluid-receiving chamber located therein that receives a bodily fluid through an inlet. Manual actuation of the actuation member relative to the actuation body moves the suction inducing member thereby increasing a volume of the suction chamber and reducing a pressure within the suction chamber. The reduction in pressure is communicated through the inlet of the fluid-receiving body for removing bodily fluid from the body cavity.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a drainage system including a fluid collection apparatus, according to one or more embodiments shown and described herein;

FIG. 2 is a diagrammatic section view of the fluid collection apparatus of FIG. 1, according to one or more embodiments shown and described herein;

FIG. 3 is a diagrammatic detail section view of an actuation body of the fluid collection apparatus of FIG. 2, according to one or more embodiments shown and described herein;

FIG. 4 illustrates a method of using the fluid collection apparatus of FIG. 2, according to one or more embodiments shown and described herein;

FIG. 5 is a diagrammatic section view of another fluid collection apparatus, according to one or more embodiments shown and described herein;

FIG. 6 is another diagrammatic section view of the fluid collection apparatus of FIG. 5, according to one or more embodiments shown and described herein;

FIG. 7 is a diagrammatic section view of another fluid collection apparatus, according to one or more embodiments shown and described herein;

FIG. 8 is a diagrammatic view of another fluid collection apparatus, according to one or more embodiments shown and described herein; and

FIG. 9 is a section view of the fluid collection apparatus of FIG. 9, according to one or more embodiments shown and described herein.

### DETAILED DESCRIPTION

Embodiments described herein are generally directed to drainage systems for draining bodily fluids from body cavities. The drainage systems may include a fluid collection apparatus that includes a housing including a fluid-receiving body having a fluid-receiving chamber that receives bodily fluid through an inlet that is in communication with the fluid-receiving chamber. The fluid collection apparatus may further include an actuation body that is connected to the fluid-receiving body. In some embodiments, the fluid-receiving body and the actuation body are parts of the same housing. The actuation body includes an actuation member that is accessible from outside the actuation body and is manually movable relative to the actuation body along a dimension of the actuation body. The actuation body is operatively connected to a suction inducing member. As will be described in greater detail herein, manual actuation of the actuation member relative to the actuation body moves the suction inducing member a selected distance thereby increasing a volume of a suction chamber thereby reducing a pressure within the suction chamber a selected amount. The reduced pressure is communicated through the inlet of the fluid-receiving body that can be communicated to an implanted drainage catheter via a drainage line that is connected to the inlet.

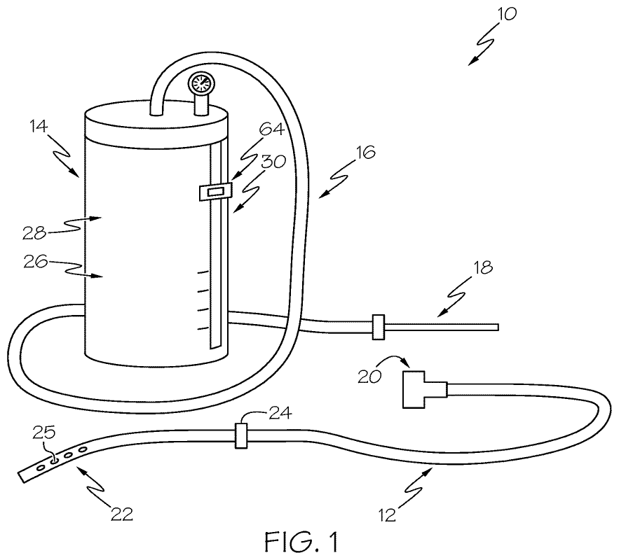

Referring to FIG. 1, a drainage system 10 for use in draining bodily fluids from body cavities includes a drainage catheter 12 that may be partially implanted in a patient's body and a fluid collection apparatus 14 that is fluidly connected to the drainage catheter 12. The fluid collection apparatus 14 may be connected to the drainage catheter 12 by a drainage line 16 that extends from the fluid collection apparatus 14 to the drainage catheter 12. In some embodiments, the drainage line 16 may include a connector 18 that connects to a one-way valve 20. As an example, the one-way valve 20 may be a two-piece valve that is normally closed, but that opens with insertion of the connector 18 to allow bodily fluid to flow through the drainage line 16. A distal end portion 22 of the drainage catheter 12 may be implanted in the patient's body for pleural or peritoneal drainage or other body cavity drainage. In some embodiments, the distal end portion 22 may be distal of a sealing cuff 24 and include fenestrations 25 through which the bodily fluid may enter the drainage catheter 12 and be carried from the pleural, peritoneal or other cavity space. The fluid collection apparatus 14 supplies negative pressure through the drainage line 16 and the drainage catheter 12 thereby providing a suction to automatically draw bodily fluid from the cavity in which the drainage catheter 12 is inserted. As used herein, the terms “negative pressure,” “suction,” vacuum” and “partial vacuum” may be used interchangeably and refer to a force over an area produced by a difference in pressure. Pressure may be referred to as “negative” in reference to atmospheric pressure (i.e., gauge pressure). The fluid collection apparatus 14 includes an outer housing 26 that is formed by a fluid-receiving body 28 and an actuation body 30. The fluid-receiving body 28 is where the bodily fluid is collected and the actuation body 30 includes an actuation member 64 that is used to generate a partial vacuum to initiate and provide fluid drainage from the body cavity.

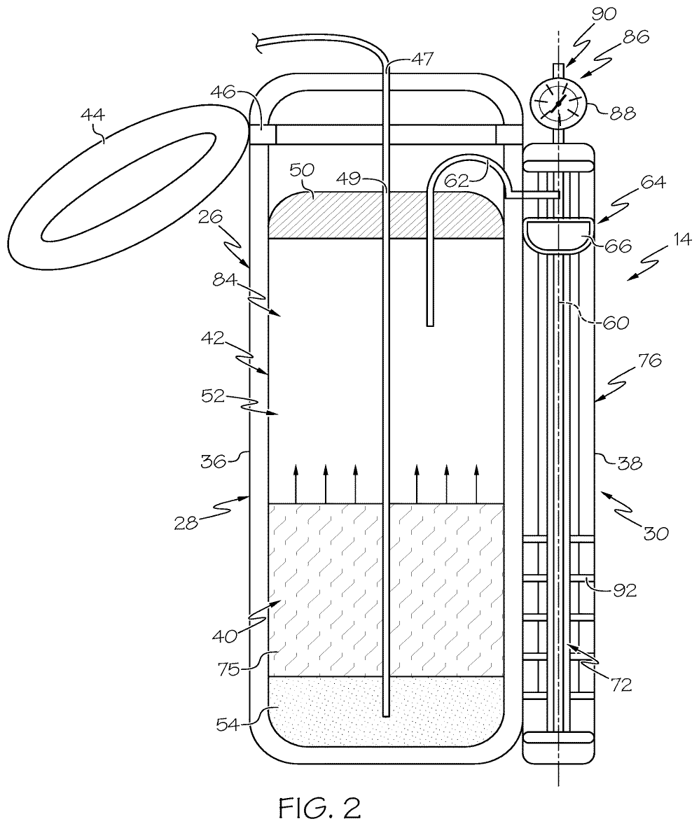

FIG. 2 illustrates a diagrammatic section view of fluid collection apparatus 14. The fluid-receiving body 28 and the actuation body 30 together form the outer housing 26. In the illustrated example, the fluid-receiving body 28 and the actuation body 30 each include their own body housing 36 and 38 that are connected together to form the outer housing 26. The body housing 36 defines a first fluid-receiving chamber 40 that is located in the fluid-receiving body 28. The first fluid-receiving chamber 40 is sized to receive a fluid-receiving container 42 (e.g., a fixed volume bottle, such as formed by glass or plastic). In some embodiments, the fluid-receiving body 28 may include a lid 44 that can be removed so that the fluid-receiving container 42 can be placed into the first fluid-receiving chamber 40. The lid 44 may be removed and replaced so that new fluid-receiving containers 42 can be removed and replaced. A seal 46 may be provided between the lid 44 and the fluid-receiving body 28 to provide a fluid-tight seal therebetween.

The fluid-receiving container 42 may be sealed using any suitable seal 50 and have a second fluid-receiving chamber 52. Thus, with the fluid-receiving container 42 located in the first fluid receiving-chamber 40 of the fluid-receiving body 28, there are nested second and first fluid-receiving chambers 52 and 40 and the bodily fluid 54 is received by both the second fluid-receiving chamber 52 and the first fluid-receiving chamber 40 simultaneously through inlets 47 and 49 into the fluid-receiving body 28 and second fluid-receiving chamber 52, respectively. A liquid 75, such as saline may be provided in the sealed fluid-receiving container 42.

The actuation body 30 is located alongside the fluid-receiving body 28 and includes a suction chamber 60 located therein. The suction chamber 60 is at least partially sealed to prevent entry of outside air into the suction chamber 60 or a portion thereof during use. The suction chamber 60 is in communication with the fluid-receiving chamber 52 via a conduit 62. The conduit 62 may be formed by any suitable tubular structure, such as a pipe, hollow needle, tubing, etc.

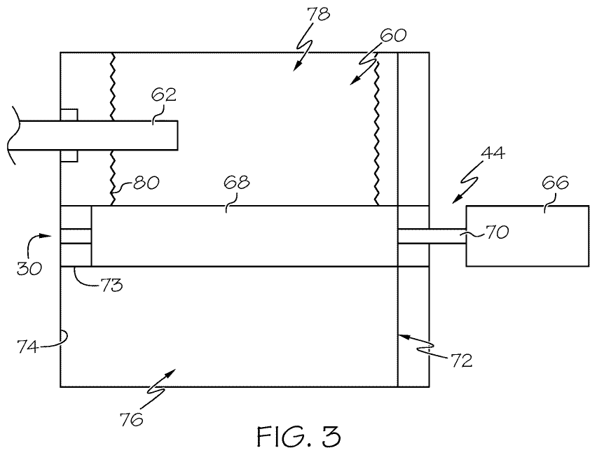

Referring also to FIG. 3, the actuation body 30 includes an actuation member 64 that is accessible from outside the actuation body 30. The actuation member 64 includes an enlarged actuation head 66 and is connected to a suction inducing member 68 via a stem-like connection 70 that extends through a slot 72 through the actuation body 30. The suction inducing member 68 of FIG. 3 may perform similar to a barrel seal of a syringe and includes one or more seal members 73 that seal against an inner surface 74 around the suction chamber 60. In other embodiment, the suction inducing member 68 may not itself seal against inner surface 74 and another type of seal may be used, such as a diaphragm that seals against the inner surface 74. As the suction inducing member 68 is moved away from an end of the conduit 62 along barrel portion 76, a volume 78 of the suction chamber 60 increases thereby reducing the pressure inside the suction chamber 60. A seal 80, such as a diaphragm, bellow, etc. may be used to isolate the suction chamber 60 from the environment. The seal may be formed of a flexible material (e.g., rubber, plastic, etc.) to expand and contract with movement of the suction inducing member 68.

It can be appreciated that increasing the volume of the suction chamber 60 acts as a suction that is communicated to the fluid-receiving chamber 52 of the fluid-receiving container 42 through the conduit 62. A portion of a volume of the fluid-receiving chamber 52 (e.g., at least about 10 percent, such as at least about 20 percent, such as at least about 30 percent, such as at least about 40 percent, such as at least about 50 percent, such as at least about 60 percent, such as at least about 70 percent, such as between about 10 percent and about 70 percent) is pre-filled with the fluid 75, such as saline, thereby reducing the air-filled volume of the fluid-receiving chamber 52. In some embodiments, the suction chamber 60 may be capable of expanding to a maximum volume that is a pre-selected percentage of the non-filled volume 84 of the fluid-receiving chamber 52 of the fluid-receiving container 42 prior to a drainage procedure. For example, the pre-selected percentage may be at least about 10 percent, such as at least about 20 percent, such as at least about 30 percent, such as at least about 40 percent, such as at least about 50 percent, such as at least about 60 percent, such as at least about 70 percent, such as between about 10 percent and about 70 percent of the non-filled volume 84 of the fluid-receiving chamber 52. The maximum volume of the suction chamber 60 is selected to reduce a pressure within the fluid-receiving chamber 52 that is capable of initiating a drainage procedure.

A pressure gauge 86 may be provided to provide a pressure measurement inside the suction chamber 60. In some embodiments, the pressure gauge 86 may be connected to the actuation body 30 and be visible outside the actuation body 30. A relief valve 88 may be provided to allow air to enter the suction chamber 60 and reduce the pressure within the fluid-receiving chamber 52. A button 90 or other input element may be provided to allow for manual opening of the relief valve 88.

In some embodiments, the actuation body 30 may include graduations 92 or other suitable indication of volume size of and/or pressure within the suction chamber 60. The graduations 92 provide a visual indication of location of the actuation member 64 and corresponding size and pressure of the suction chamber 60. In some embodiments, the graduations may not include a pressure and/or volume size marking, but may still provide a reference for a user based on previous uses and settings.

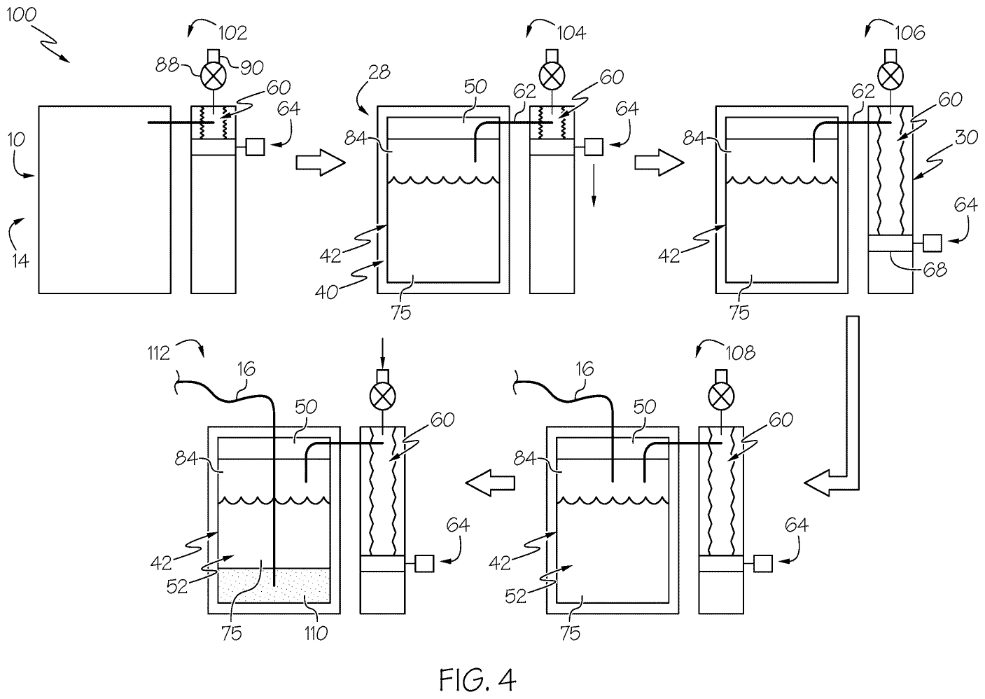

FIG. 4 diagrammatically illustrates a bodily fluid drainage method 100 using the drainage system 10 including fluid collection apparatus 14. At step 102, the fluid collection apparatus 14 is positioned for operation. The actuation member 64 may be placed at a minimum volume position. In some embodiments, the button 90 may be pressed to open the relief valve 88 and release air as the actuation member 64 is moved to the minimum volume position. Opening the relief valve 88 can allow air to escape to facilitate formation of the partial vacuum once the suction chamber 60 is expanded. At step 104, a fluid-receiving container 42 is placed inside the fluid-receiving chamber 40 of the fluid-receiving body 28. With the fluid-receiving container 42 in the fluid-receiving body 28, an end of the conduit 62 is pierced through the seal 50 of the fluid-receiving container 42. The end of the conduit 62 may be beveled or otherwise shaped into a point to facilitate piercing through the seal 50 in order to provide fluid communication between the suction chamber 60 and the fluid-receiving chamber 52. In some embodiments, the end of the conduit 62 may be formed by an instrument, such as a hollow needle, spike, etc. As discussed above, the fluid-receiving chamber 52 may be partially filled with the fluid 75, such as saline or a saline solution. The conduit 62 is placed in the non-filled volume 84.

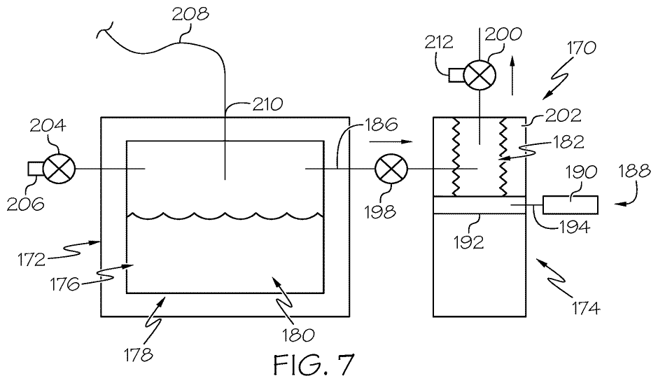

At step 106, the actuation member 64 is moved by a user relative to the actuation body 30 to an increased volume position. Manual actuation of the actuation member 64 relative to the actuation body 30 moves the suction inducing member 68 thereby increasing the volume of the suction chamber 60 and reducing a pressure in the suction chamber 60. Because the non-filled volume 84 of the fluid-receiving container 42 is in fluid communication with the suction chamber 60 via the conduit 62, the overall volume of the non-filled volume 84 plus the suction chamber 60 is increased and pressure is reduced as given by the ideal gas law. In some embodiments, the actuation member may behave as a pump, as shown and described with reference to FIG. 7.

With the reduced pressure in the combined fluid-receiving chamber 52 and suction chamber 60, an end of the drainage line 16 is pierced through the seal 50 of the fluid-receiving container 42 at step 108. The end of the drainage line 16 may be beveled or otherwise shaped into a point to facilitate piercing through the seal 50 in order to provide fluid communication between the suction chamber 60 and the fluid-receiving chamber 52. In some embodiments, the end of the drainage line 16 may be formed by an instrument, such as a hollow needle, spike, etc. In some examples, the position of the actuation member 64 may be adjusted to a user-selected position between the maximum volume position and the minimum volume position to increase or reduce the negative pressure communicated through the drainage line 16. The adjustment in negative pressure can be user-selected based on feel of the amount of suction in the body cavity.

As the end of the drainage line 16 passes through the non-filled volume 84 at step 108, the reduced pressure is communicated through the drainage line 16, to the drainage catheter 12 already connected thereto and into the body cavity. The difference in pressure causes bodily fluid 110 to be drawn into the drainage catheter 12, through the drainage line 16 and deposited into the fluid-receiving chamber 52. In the illustrated embodiment, the end of the drainage line 16 is located beneath the fluid 75 where the fluid 75 provides a barrier between the bodily fluid 110 and the non-filled volume 84. The fluid collection apparatus 14 may be placed in a low location, such as on the floor, such that drainage can continue under the influence of gravity. Once the drainage operation is completed at step 112, the fluid-receiving container 42 may be removed from the fluid-receiving body 28 and disposed of. This drainage process may be repeated as frequently as needed by the user.

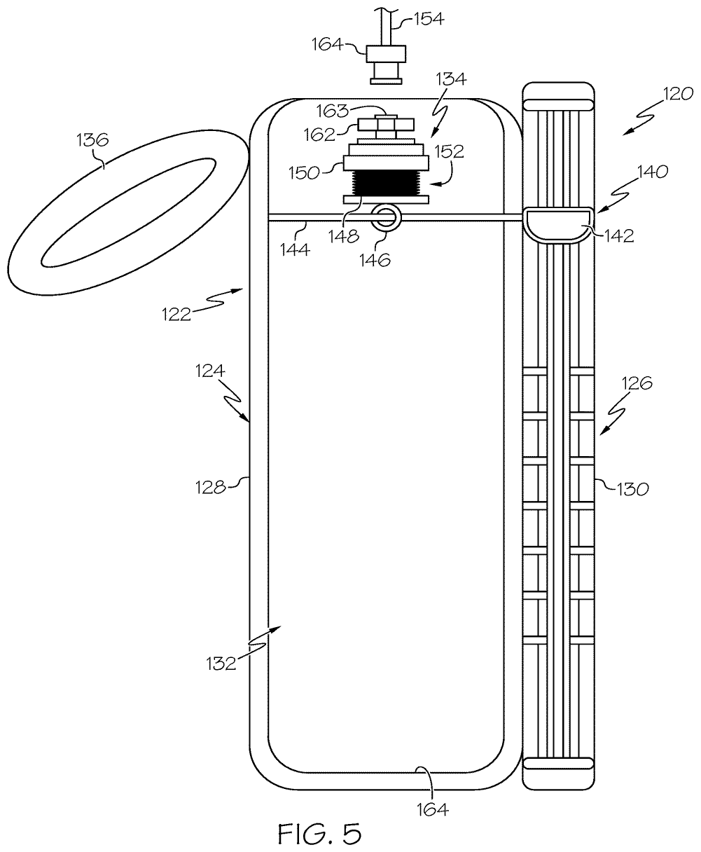

The embodiments described above uses a fluid-receiving container having a fixed volume with a separate suction chamber that expands and contracts in order to reduce and increase negative pressure within the fluid-receiving container. Referring to FIG. 5, another embodiment of a fluid collection apparatus 120 of a drainage system includes an outer housing 122 that is formed by a fluid-receiving body 124 and an actuation body 126. The fluid-receiving body 124 and the actuation body 126 together form the outer housing 122. In the illustrated example, the fluid-receiving body 124 and the actuation body 126 each include their own body housing 128 and 130 that are connected together to form the outer housing 122. The body housing 128 defines a first fluid-receiving chamber 132 that is located in the fluid-receiving body 124. The first fluid-receiving chamber 132 is sized to receive a fluid-receiving container 134 (e.g., a collapsible and expandable bottle). In some embodiments, the fluid-receiving body 124 may include a lid 136 that can be removed so that the fluid-receiving container 134 can be placed into the first fluid-receiving chamber 132. As will be described, in this embodiment, the fluid-receiving container 134, itself, forms a suction chamber.

The actuation body 126 includes an actuation member 140 that is accessible from outside the actuation body 126. The actuation member 140 includes an enlarged actuation head 142 and a suction inducing member 144. In this embodiment, the suction inducing member 144 may be a connector (e.g., a rod, hook, etc.) that connects the actuation member 140 to an underside 148 of the fluid-receiving container 134. In the illustrated example, the suction inducing member 144 may be threaded through a mating connector 146 located on the underside 148 of the fluid-receiving container 134.

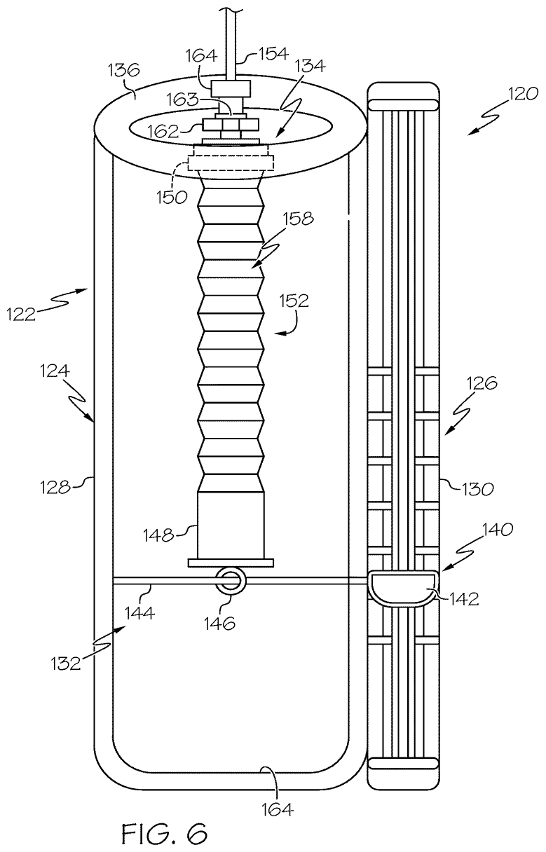

The fluid-receiving container 134 has a container body 150 that includes an expandable section 152 that, in the illustrated embodiment, is an accordion or bellow-shape. Other expandable section configurations may be used, such as use of a resiliently expandable film. If a resiliently expandable material is used, the expandable section may be biased toward a fully contracted configuration. As can be appreciated, as the actuation member 140 is moved downward relative to the actuation body 126, the suction inducing member 144 pulls on the underside of the fluid-receiving container 134 thereby expanding the expandable section 152, which acts as a suction force that is communicated through drainage line 154. In this regard, the fluid-receiving container 134 has a fully contracted configuration and a fully expanded configuration and the actuation member 140 has a minimum volume position and a maximum volume position (FIG. 6).

FIGS. 5 and 6 diagrammatically illustrate a bodily fluid drainage method using the drainage system 118 including fluid collection apparatus 120. Referring first to FIG. 5, a fluid-receiving container 134 having an expandable second fluid receiving chamber is placed within the first fluid-receiving chamber 132 of the fluid-receiving body 124. The actuation member 140 may be placed in the minimum volume position and the mating connector 146 may be threaded onto the suction inducing member 144. In some embodiments, the fluid-receiving body 124 may include support structure 162 that is used to support the fluid-receiving container 134 spaced from a bottom 164 of the fluid-receiving body 124 in order to provide a space that allows the fluid-receiving container 134 to expand (FIG. 6). In some embodiments, the fluid-receiving container 134 is provided to the user in the fully contracted configuration. In other embodiments, the user may manually contract the fluid-receiving container 134 and evacuate the air therein before threading the mating connector 146 onto the suction inducing member 144.

The actuation member 140 is moved by a user relative to the actuation body 126 to an increased volume position. Manual actuation of the actuation member 140 relative to the actuation body 126 moves the suction inducing member 144 thereby increasing the volume of the suction chamber 158 and reducing a pressure in the suction chamber 158. Because the volume of the suction chamber 158 is increased, the pressure is reduced as given by the ideal gas law.

The drainage line 154 includes a connector 161 that is connected to a mating connector 163 of the fluid-receiving container 134. The drainage line 154 may already be connected to a drainage catheter as described above. The drainage line 154 may be connected to the drainage catheter before or after expanding the fluid-receiving container 134. In some examples, the position of the actuation member 140 may be adjusted to increase or reduce the negative pressure communicated through the drainage line 154. The adjustment in negative pressure can be user-selected based on feel of the amount of suction in the body cavity.

The fluid collection apparatus 120 may be placed in a low location, such as on the floor, such that drainage can continue under the influence of gravity. Once the drainage operation is completed, the fluid-receiving container 134 may be removed from the fluid-receiving body 124 and disposed of.

Referring to FIG. 7, another embodiment of a fluid collection apparatus 170 pumps air out of a fluid-receiving container 176 and is similar to the fluid collection apparatus of FIG. 2. The fluid collection apparatus 170 includes a fluid-receiving body 172 and an actuation body 174. Again, the fluid-receiving body 172 and the actuation body 174 may together form a same outer housing. The fluid-receiving container 176 is located in a fluid-receiving chamber 178 of the fluid-receiving body 172. The fluid-receiving container 176 has a fluid-receiving chamber 180 that may be partially pre-filled with a fluid, such as saline, as described above.

The actuation body 174 is located alongside the fluid-receiving body 172 and includes a suction chamber 182 located therein. The suction chamber 182 is at least partially sealed to prevent entry of outside air into the suction chamber 182 or a portion thereof during use. The suction chamber 182 is in communication with the fluid-receiving chamber 180 via a conduit 186.

The actuation body 174 includes an actuation member 188 that is accessible from outside the actuation body 174. The actuation member 188 includes an enlarged actuation head 190 and is connected to a suction inducing member 192 via a stem-like connection 194 that extends through a slot 196 through the actuation body 174. In this embodiment, two one-way valves 198 and 200 are provided. In this example, valve 198 allows air to exit the fluid-receiving chamber 180, but not enter the fluid-receiving chamber 180. Valve 200 allows air to exit the suction chamber 182, but not enter the suction chamber 182. A seal 202 (e.g., a diaphragm, bellows, etc.) may be used to seal the suction chamber 184, as described above. Another valve 204 may be provided to allow entry of air into the fluid-receiving chamber 180 using release button 206 that opens the valve 204 after a drainage operation. A drainage line 208 extends through an inlet 210 of the fluid-receiving body 172. In operation, the actuation member 188 may be moved in a reciprocal fashion in order to repeatedly expand and contract the suction chamber 184 thereby pumping air out of the fluid-receiving chamber 180 thereby reducing a pressure in the fluid-receiving chamber 180 for a drainage operation. Release buttons 206 and 212 may be used to allow air to enter the fluid-receiving chamber 180 and the suction chamber 182 upon user actuation.

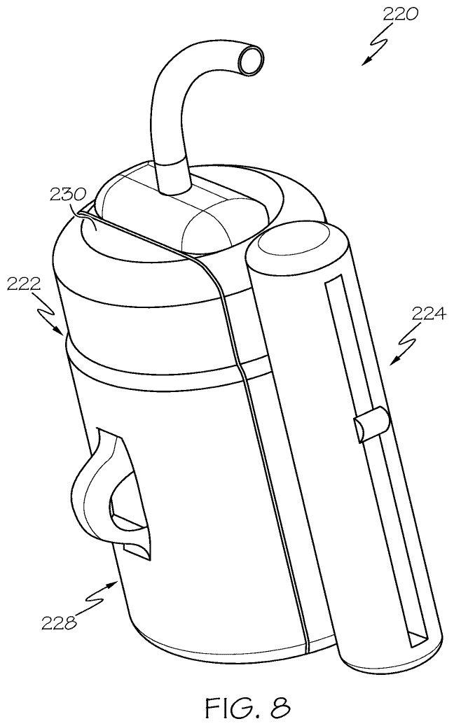

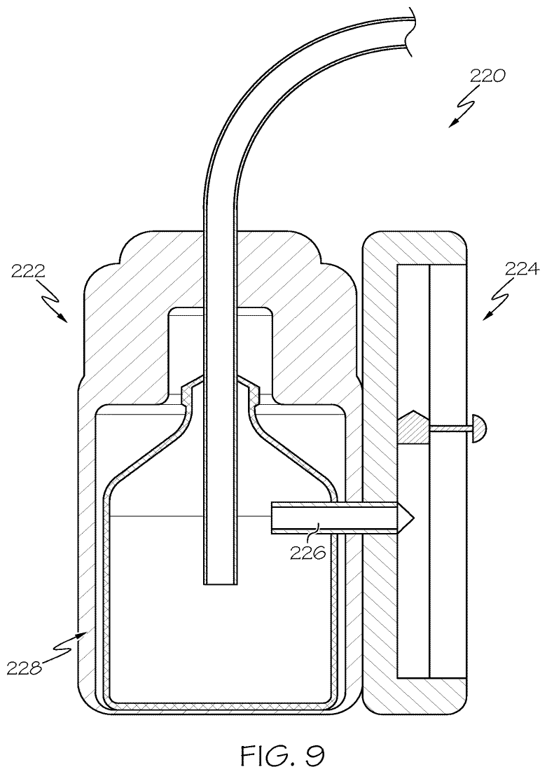

Referring briefly to FIGS. 8 and 9, another embodiment of a fluid collection apparatus 220 is similar to the fluid-collection apparatus of FIG. 2. In this example, a fluid-receiving body 222 and actuation body 224 are formed separately and then connected together via conduit 226 (FIG. 9) to form an outer housing 228. The fluid-receiving body 222 includes a removable section 230 that can be used to provide access to a fluid-receiving chamber in order to place a fluid-receiving body 222.

The above-described fluid collection apparatuses may be suitable for pleural and peritoneal drainage operations and include a fluid-receiving body and an actuation body that together can form a single outer housing. The fluid-receiving body has a fluid-receiving chamber that receives a separate fluid-receiving container therein. The actuation body includes an actuation member that is movable along a dimension of the actuation body. The actuation member is used to expand and contract a suction chamber that can be used to induce a partial vacuum in the fluid-receiving container. The partial vacuum can be communicated through a drainage line to an indwelling drainage catheter to initiate drainage of bodily fluid. The actuation member allows for adjusting the magnitude of the negative pressure so that a user can control the amount of suction force that is applied to initiate drainage. Once drainage is initiated, the drainage may continue under the force of gravity alone, at least for a time, until the suction chamber is again expanded using the actuation member. The actuation member can also allow for fine adjustment by the user of the suction force.

In some embodiments, the fluid-receiving body and the actuation body are part of the same container, which can reduce the number of separate components needed for a drainage procedure. The fluid-receiving container may also be removable from the fluid-receiving body, which can allow the fluid collection apparatus to be reusable simply by replacing the fluid-receiving container.

Embodiments can be described with reference to the following numbered clauses, with certain features laid out in the dependent clauses:
Clause 1: A drainage system for draining bodily fluid from a body cavity, the drainage system comprising: a fluid-receiving body having a fluid-receiving chamber located therein that receives a bodily fluid through an inlet; and an actuation body connected to the fluid-receiving body, the actuation body comprising an actuation member that is accessible from outside the actuation body and is manually movable relative to the actuation body along a dimension of the actuation body, the actuation member operatively connected to a suction inducing member; wherein manual actuation of the actuation member relative to the actuation body moves the suction inducing member thereby increasing a volume of a suction chamber and reducing a pressure within the suction chamber, the reduced pressure communicated through the inlet of the fluid-receiving body.
Clause 2: The drainage system of clause 1, wherein the fluid-receiving body and the actuation body are part of a same outer container.
Clause 3: The drainage system of any of the above clauses further comprising a fluid-receiving container of fixed volume that is located in the fluid-receiving chamber, the fluid-receiving container receiving bodily fluid entering the fluid-receiving chamber through the inlet.
Clause 4: The drainage system of clause 3, wherein the fluid-receiving container is removable from the fluid-receiving chamber.
Clause 5: The drainage system of clause 3 or 4, wherein the fluid-receiving body comprises a removable section that allows access to the fluid receiving container.
Clause 6: The drainage system of clause 3, 4 or 5, wherein the fixed volume of the fluid-receiving container is in communication with the suction chamber to reduce pressure within the fixed volume of the fluid-receiving container.
Clause 7: The drainage system of any of the above clauses, wherein movement of the suction inducing member expands the volume of the suction chamber without allowing air from the surroundings into the suction chamber such that pressure is reduced within the suction chamber.
Clause 8: The drainage system of any of the above clauses, wherein the suction chamber is part of a passageway through the actuation body along which the suction inducing member travels, the suction inducing member comprising a seal that seals against an outer wall of the passageway as the suction inducing member moves along the passageway.
Clause 9: The drainage system of clause 8, wherein a volume of the passageway is fixed, the suction chamber located between the suction inducing member and a closed end of the passageway.
Clause 10: The drainage system of clause 1 or 2 further comprising a fluid-receiving container comprising a bellows having the suction chamber, the suction inducing member connecting the actuation member to the fluid-receiving container such that movement of the actuation member expands the bellows without allowing air into the suction chamber such that pressure is reduced within the suction chamber.
Clause 11: The drainage system of clause 10, wherein the fluid-receiving container is located in the fluid-receiving chamber, the suction chamber receiving bodily fluid entering the fluid-receiving chamber through the inlet.
Clause 12: A method of forming a drainage system including a suction chamber configured to provide reduced pressure that is communicated to a body cavity for removing bodily fluids from the body cavity, the method comprising: forming an actuation body and a fluid-receiving body, the fluid-receiving body having a fluid-receiving chamber located therein that receives a bodily fluid through an inlet; and movably connecting an actuation member and a suction inducing member connected thereto to the actuation body; wherein manual actuation of the actuation member relative to the actuation body moves the suction inducing member thereby increasing a volume of the suction chamber and reducing a pressure within the suction chamber, the reduction in pressure being communicated through the inlet of the fluid-receiving body for removing bodily fluid from the body cavity.
Clause 13: The method of clause 12, wherein the fluid-receiving body and the actuation body are part of a same outer container
Clause 14: The method of clause 12 or 13 further comprising locating a fluid-receiving container of fixed volume in the fluid-receiving chamber, the fluid-receiving container receiving bodily fluid entering the fluid-receiving chamber through the inlet.
Clause 15: The method of any of clause 14 further comprising removing the fluid-receiving container from the fluid-receiving chamber.
Clause 16: The method of clause 14 or 15, wherein the fluid-receiving body comprises a removable section that allows access to the fluid receiving container, the method further comprising separating the removable section from the fluid-receiving body.
Clause 17: The method of clauses 14 to 16 further comprising providing fluid communication between the fixed volume of the fluid-receiving container and the suction chamber to reduce pressure within the fixed volume of the fluid-receiving container.
Clause 18: The method of clauses 12 to 17, wherein movement of the suction inducing member expanding the volume of the suction chamber without allowing air from the surroundings into the suction chamber such that pressure thereby reducing within the suction chamber.
Clause 19: The method of clauses 12 to 18, wherein the suction chamber is part of a passageway through the actuation body along which the suction inducing member travels, the suction inducing member comprising a seal sealing against an outer wall of the passageway as the suction inducing member moves along the passageway.
Clause 20: The method of clause 19, wherein a volume of the passageway is fixed, the suction chamber located between the suction inducing member and a closed end of the passageway.
Clause 21: The method of clause 12 further comprising a fluid-receiving container comprising a bellows having the suction chamber, the suction inducing member connecting the actuation member to the fluid-receiving container such that movement of the actuation member expands the bellows without allowing air into the suction chamber such that pressure is reduced within the suction chamber.
Clause 22: The method of clause 21 comprising locating the fluid-receiving container in the fluid-receiving chamber, the suction chamber receiving bodily fluid entering the fluid-receiving chamber through the inlet.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A drainage system for draining bodily fluid from a body cavity, the drainage system comprising:
a fluid-receiving body having a fluid-receiving chamber located therein that receives a bodily fluid through an inlet; and
an actuation body connected to the fluid-receiving body, the actuation body comprising an actuation member that is accessible from outside the actuation body and is manually movable relative to the actuation body along a dimension of the actuation body, the actuation member operatively connected to a suction inducing member;
wherein manual actuation of the actuation member relative to the actuation body moves the suction inducing member thereby increasing a volume of a suction chamber and reducing a pressure within the suction chamber, the reduced pressure communicated through the inlet of the fluid-receiving body.

2. The drainage system of claim 1, wherein the fluid-receiving body and the actuation body are part of a same outer container.

3. The drainage system of claim 1 further comprising a fluid-receiving container of fixed volume that is located in the fluid-receiving chamber, the fluid-receiving container receiving bodily fluid entering the fluid-receiving chamber through the inlet.

4. The drainage system of claim 3, wherein the fluid-receiving container is removable from the fluid-receiving chamber.

5. The drainage system of claim 4, wherein the fluid-receiving body comprises a removable section that allows access to the fluid receiving container.

6. The drainage system of claim 3, wherein the fixed volume of the fluid-receiving container is in communication with the suction chamber to reduce pressure within the fixed volume of the fluid-receiving container.

7. The drainage system of claim 1, wherein movement of the suction inducing member expands the volume of the suction chamber without allowing air from the surroundings into the suction chamber such that pressure is reduced within the suction chamber.

8. The drainage system of claim 1, wherein the suction chamber is part of a passageway through the actuation body along which the suction inducing member travels, the suction inducing member comprising a seal that seals against an outer wall of the passageway as the suction inducing member moves along the passageway.

9. The drainage system of claim 8, wherein a volume of the passageway is fixed, the suction chamber located between the suction inducing member and a closed end of the passageway.

10. The drainage system of claim 1 further comprising a fluid-receiving container comprising a bellows having the suction chamber, the suction inducing member connecting the actuation member to the fluid-receiving container such that movement of the actuation member expands the bellows without allowing air into the suction chamber such that pressure is reduced within the suction chamber.

11. The drainage system of claim 10, wherein the fluid-receiving container is located in the fluid-receiving chamber, the suction chamber receiving bodily fluid entering the fluid-receiving chamber through the inlet.

12. A method of forming a drainage system comprising a suction chamber configured to provide reduced pressure that is communicated to a body cavity for removing bodily fluids from the body cavity, the method comprising:
forming an actuation body and a fluid-receiving body, the fluid-receiving body having a fluid-receiving chamber located therein that receives a bodily fluid through an inlet; and
movably connecting an actuation member and a suction inducing member connected thereto to the actuation body;
wherein manual actuation of the actuation member relative to the actuation body moves the suction inducing member thereby increasing a volume of the suction chamber and reducing a pressure within the suction chamber, the reduction in pressure being communicated through the inlet of the fluid-receiving body for removing bodily fluid from the body cavity.

13. The method of claim 12, wherein the fluid-receiving body and the actuation body are part of a same outer container.

14. The method of claim 12 further comprising locating a fluid-receiving container of fixed volume in the fluid-receiving chamber, the fluid-receiving container receiving bodily fluid entering the fluid-receiving chamber through the inlet.

15. The method of claim 14 further comprising removing the fluid-receiving container from the fluid-receiving chamber.

16. The method of claim 15, wherein the fluid-receiving body comprises a removable section that allows access to the fluid receiving container, the method further comprising separating the removable section from the fluid-receiving body.

17. The method of claim 14 further comprising providing fluid communication between the fixed volume of the fluid-receiving container and the suction chamber to reduce pressure within the fixed volume of the fluid-receiving container.

18. The method of claim 12, wherein movement of the suction inducing member expanding the volume of the suction chamber without allowing air from the surroundings into the suction chamber such that pressure thereby reducing within the suction chamber.

19. The method of claim 12, wherein the suction chamber is part of a passageway through the actuation body along which the suction inducing member travels, the suction inducing member comprising a seal sealing against an outer wall of the passageway as the suction inducing member moves along the passageway.

20. The method of claim 19, wherein a volume of the passageway is fixed, the suction chamber located between the suction inducing member and a closed end of the passageway.

21. The method of claim 12 further comprising a fluid-receiving container comprising a bellows having the suction chamber, the suction inducing member connecting the actuation member to the fluid-receiving container such that movement of the actuation member expands the bellows without allowing air into the suction chamber such that pressure is reduced within the suction chamber.

22. The method of claim 21 comprising locating the fluid-receiving container in the fluid-receiving chamber, the suction chamber receiving bodily fluid entering the fluid-receiving chamber through the inlet.
